(19) United States
(12) Patent Application Publication
(10) Pub. No.: US 2025/0295675 A1
(43) Pub. Date: Sep. 25, 2025

(54) COMPOSITIONS COMPRISING HEMP EXTRACT, CONSUMABLES COMPRISING HEMP EXTRACT, AND METHODS OF MITIGATING HEMP FLAVOR THEREOF

(71) Applicant: University of Vermont and State Agricultural College, Burlington, VT (US)

(72) Inventors: Mingruo Guo, Burlington, VT (US); Kalev Freeman, Burlington, VT (US); Eric Kawka, Hardwick, VT (US); William Cats-Baril, Charlotte, VT (US); Hao Shi, Burlington, VT (US); Monique McHenry, Duxbury, VT (US)

(21) Appl. No.: 18/869,603

(22) PCT Filed: May 31, 2023
(86) PCT No.: PCT/US2023/067675

§ 371 Date: Nov. 26, 2024

Related U.S. Application Data
(60) Provisional application No. 63/347,698, filed on Jun. 1, 2022.

(51) Int. Cl. A61K 31/00; A61K 31/724

(52) U.S. Cl. CPC A61K 31/658; A61K 31/724

(57) ABSTRACT

Compositions and consumables that include hemp extract in a manner that promotes bioavailabilty of the hemp extract constituents are described. Techniques for incorporating hemp extract into a composition or consumable, such that the hemp flavor of the composition or consumable may be mitigated, are also described. Bioavailability promotion and hemp flavor mitigation may be achieved by encapsulating the hemp extract using β-cyclodextrin and at least one of whey protein or polymerized whey protein.

### RELATED APPLICATION

The present application claims the benefit under 35 U.S.C. § 119(e) to U.S. Provisional Application No. 63/347,698 filed on Jun. 1, 2022, the contents of which is incorporated by reference herein in its entirety.

### BACKGROUND

Cannabis is a flowering plant that may be classified by its intoxicating and non-intoxicating constituents. Plants producing an abundance of intoxicating constituents are often referred to as marijuana, whereas those with non-intoxicating constituents are referred to as hemp.

Oils extractable from hemp include non-intoxicating cannabinoids, flavonoids, and terpenes. Each of these non-intoxicating oils have therapeutic potential.

Hemp oils and extracts are available as tinctures and in various commercial products.

### SUMMARY

Aspects of the present disclosure relate compositions and consumables that include hemp extract in a manner that promotes bioavailabilty of the hemp extract constituents. Moreover, aspects of the present disclosure involve incorporating hemp extract into a composition or consumable such that the hemp flavor of the composition or consumable may be mitigated. In at least some embodiments, the foregoing bioavailability promotion and hemp flavor mitigation may be achieved by encapsulating the hemp extract using β-cyclodextrin and at least one of whey protein or polymerized whey protein.

A first aspect of the present disclosure relates to a composition comprising hemp extract and hemp extract encapsulate, where the hemp extract comprises cannabidiol (CBD) and terpenes, and the hemp extract encapsulate comprises β-cyclodextrin and at least one of whey protein and polymerized whey protein.

In some embodiments of the first aspect, the β-cyclodextrin and whey protein/polymerized whey protein are present in a ratio of about 3:1 w/w.

In some embodiments of the first aspect, the β-cyclodextrin and whey protein/polymerized whey protein are present in a ratio of about 1:1 w/w.

In some embodiments of the first aspect, the whey protein/polymerized whey protein and β-cyclodextrin are present in a ratio of about 3:1 w/w to about 5:1 w/w.

In some embodiments of the first aspect, the hemp extract and hemp extract encapsulate are present in a ratio of about 1:5 w/w.

In some embodiments of the first aspect, the β-cyclodextrin and whey protein/polymerized whey protein are configured to increase bioavailability of the CBD.

In some embodiments of the first aspect, the β-cyclodextrin and whey protein/polymerized whey protein are configured to mask the flavor of the terpenes.

In some embodiments of the first aspect, the hemp extract comprises about 30 wt % to about 60 wt % CBD.

In some embodiments of the first aspect, the hemp extract encapsulate comprises at least one of whey protein isolate and polymerized whey protein isolate.

A second aspect of the present disclosure relates to a consumable composition comprising a composition of the first aspect of the present disclosure.

In some embodiments of the second aspect, the consumable composition may comprise a dairy component.

In some embodiments of the second aspect, the consumable composition is selected from the group consisting of ice cream, yogurt, frozen yogurt, whipped cream, and cheese.

In some embodiments of the second aspect, the consumable composition is selected from the group consisting of a powder, a shake, a tablet, and a capsule.

In some embodiments of the second aspect, the consumable composition is dog food.

A third aspect of the present disclosure relates to a method of preparing ice cream comprising hemp extract, where the method comprises preparing or obtaining milk; preparing a sugar syrup comprising stabilizer, emulsifier, and sugar in water; heating the sugar syrup to produce a heated sugar syrup; preparing a first mixture by mixing the milk with the heated sugar syrup; preparing a second mixture by mixing the first mixture with β-cyclodextrin; preparing or obtaining a third mixture comprising hemp extract and heavy cream; heating the third mixture to produce a heated third mixture; preparing a fourth mixture by mixing the heated third mixture with at least one of whey protein and polymerized whey protein; and producing the ice cream mixture by mixing the second mixture with the fourth mixture.

In some embodiments of the third aspect, preparing the milk comprises dissolving milk powder in water.

In some embodiments of the third aspect, the stabilizer comprises guar gum, carob bean gum, cellulose gum, and combinations thereof.

In some embodiments of the third aspect, the emulsifer comprises egg yolk, soy lecithin, monogliceride, digliceride, and combinations thereof.

In some embodiments of the third aspect, producing the heated sugar syrup comprises heating the sugar syrup to about 85° C.

In some embodiments of the third aspect, producing the heated third mixture comprises heating the third mixture to about 75° C.

In some embodiments of the third aspect, the whey protein/polymerized whey protein and β-cyclodextrin are present in the ice cream in a ratio of about 1:3 w/w.

In some embodiments of the third aspect, the whey protein/polymerized whey protein and β-cyclodextrin are present in the ice cream in a ratio of about 1:1 w/w.

In some embodiments of the third aspect, the whey protein/polymerized whey protein and β-cyclodextrin are present in the ice cream in a ratio of about 3:1 w/w to about 5:1 w/w.

In some embodiments of the third aspect, the hemp extract is present in the ice cream in a ratio of about 1:5 w/w with respect to the whey protein/polymerized whey protein and β-cyclodextrin.

### BRIEF DESCRIPTION OF THE DRAWINGS

FIG. 1 includes plots showing changes in pH and titratable acid of goat and cow milk yogurt with different treatments during storage. Ctrl: control group; HE: hemp extract; PWP: polymerized whey protein; WPI: whey protein isolate.

FIG. 2 includes plots showing changes of Lactobacillus acidophilus LA-5 and Bifidobacterium BB-12 in both goat and cow milk yogurt during storage.

FIG. 3a is a scanning electron microscopy (SEM) photograph of a goat milk yogurt control.

FIG. 3b is a SEM photograph of goat milk yogurt with hemp extract.

FIG. 3c is a SEM photograph of goat milk yogurt with hemp extract and whey protein isolate.

FIG. 3d is a SEM photograph of goat milk yogurt with hemp extract and polymerized whey protein.

### DETAILED DESCRIPTION

The present disclosure provides compositions and consumables that include hemp extract in a manner that promotes bioavailabilty of the hemp extract constituents. In addition, the present disclosure provides techniques for incorporating hemp extract into a composition or consumable such that the hemp flavor of the composition or consumable may be mitigated. In at least some embodiments, the foregoing bioavailability promotion and hemp flavor mitigation may be achieved by encapsulating the hemp extract into particles or aggregates using β-cyclodextrin and at least one of whey protein or polymerized whey protein.

### Hemp Extract

As used herein, hemp extract refers to the non-intoxicating constituents of a plant of the genus Cannabis and/or the family Cannabaceae. However, a hemp extract of the present disclosure need not be produced from a plant. Rather, some or all components of a hemp extract of the present disclosure may be produced synthetically.

In some embodiments, hemp extract of the present disclosure may be produced from raw cannabis plant material, meaning the hemp extract may be produced using one or more regions of the cannabis plant. Non-limiting examples of regions of cannabis plants are stems, seeds, flowers, leaves, pistils, colas, calyxs, trichomes, buds (including dormant buds, axillary buds, and terminal buds), petiole, rachis, bract, and roots. The plant material used may be fresh, though in certain embodiments the plant material is dried, frozen, or in another preserved state. In some embodiments, the starting, raw material has not been exposed to pesticides. In certain embodiments, the raw material is obtained from organically grown cannabis.

One skilled in the art will understand that the makeup of hemp extract of the present disclosure may depend on the starting material(s). For example, though not intended to be limiting, the composition of hemp extract, including the concentration of cannabinoids and other chemicals present therein, may depend at least in part on the composition of the starting material used in the preparation of the hemp extract.

The following is an illustrative list of one or more components that may be included in hemp extract of the present disclosure.

Hemp extract of the present disclosure may include one or more cannabinoids. A “cannabinoid” is a compound capable of interacting with a cannabinoid receptor of a cell. A cannabinoid may be produced synthetically, for example, through a chemical synthetic process or by using a biological organism such as a yeast or a bacteria modified to produce the cannabinoid. Alternatively, a cannabinoid may originate from a cannabis plant. A cannabinoid may be isolated, in pure form, or in combination with other cannabinoids. Optionally, a cannabinoid may be a decarboxylated cannabinoid or otherwise heat-transformed cannabinoid, or other cannabinoid having the structure of a metabolite that has underwent metabolic transformation in the human body. Optionally, the cannabinoid may be a cannabimimetic.

Hemp extract of the present disclosure may include cannbidiol (CBD). CBD is a specific cannabinoid having the structure:

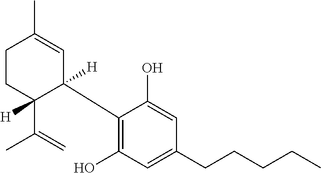 [SMILES: C=C(C)[C@@H]1CCC(C)=C[C@H]1c1c(O)cc(CCCCC)cc1O]

In some embodiments, hemp extract of the present disclosure may include up to about 60 wt % CBD. In some embodiments, hemp extract of the present disclosure may include about 30 wt % to about 60 wt % CBD.

Hemp extract of the present disclosure may include (a minimal) amount of tetrahydrocannabinol (THC). THC is a cannabinoid having the structure:

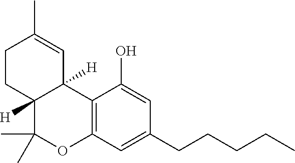 [SMILES: CCCCCc1cc(O)c2c(c1)OC(C)(C)[C@@H]1CCC(C)=C[C@@H]21]

Other cannabinoids that may be present in hemp extract include, but are not limited to: cannabigerol (CBG), cannabichromene (CBC), cannabigerivarin (CBGV), tetrahydrocannabivarin (THCV), cannabidivarin (CBDV), and cannabichromevarin (CBCV).

Hemp extract of the present disclosure may include one or more flavonoids. A flavonoid is a polyphenolic compound having a 15-carbon skeletal structure, which may include two phenyl rings and a heterocyclic ring. The chemical structure of a flavonoid may be abbreviated as C6-C3-C6. A flavonoid may be a ketone-containing compound. A flavonoid may be a flavan, isoflavonoid, flavanonol, flavanone, anthoxanthin, or anthocyanidin.

Hemp extract of the present disclosure may include one or more terpenes. Terpene is a group of volatile unsaturated hydrocarbons based on a cyclic molecule having the formula (C₆H₈)ₙ. This group includes modification of terpenes that generate terpenoids and isoprenoids. One or more terpenes may provide distinctive flavors and/or scents, and different terpenes may be used to promote different effects in humans, from relaxation and stress-relief to focus and acuity.

In some embodiments, hemp extract may be produced using supercritical carbon dioxide bio-extraction. Supercritical carbon dioxide bio-extraction is a form of supercritical fluid extraction, which separates one component (i.e., an extractant) from another (i.e., a matrix) using a supercritical fluid (e.g., carbon dioxide) as an extracting solvent. In some embodiments, the matrix may be a solid matrix. In some embodiments, the solid matrix may be dried inflorescence. As used herein, “inflorescence” refers to a complete flower head of a plant, including the stems, stalks, bracts, and flowers.

Generally, a supercritical carbon dioxide bio-extraction system may include components such as: a carbon dioxide supply, a pump, an oven comprising a heating means (a non-limiting example of which is a heating coil), an extraction cell, and a metering valve. Carbon dioxide, from the carbon dioxide supply, may be pumped to the heating means where the carbon dioxide is heated to supercritical conditions. The heated carbon dioxide may then be passed into the extraction cell. In the extraction cell, the heated carbon dioxide may diffuse into a solid matrix and dissolve cannabinoids, flavonoids, terpenes, and/or other constituents of hemp. The dissolved non-intoxicating constituents may then be passed from the extraction cell into a lower pressure area, where the non-intoxicating constituents may settle out of the heated carbon dioxide, and may be released from the metering valve as a hemp extract of the present disclosure.

In some embodiments, organic solvent-based extraction may be performed to produce a hemp extract. Art-known organic solvent-based extraction techniques may be used. A non-limiting list of organic solvents that may be used in organic solvent-based cannabinoid extraction include butane, propane, and ethanol. The low boiling point of these solvents allows extractors to remove them without risking evaporating heat-sensitive cannabinoids or terpenes. Ethanol is well suited for large-scale extractions. Butane and propane extraction technology can produce a product with lighter color and more of a terpene-rich smell.

While the foregoing describes certain extraction techniques that may be used to produce a hemp extract, the present disclosure is not limited thereto. There are various known techniques for producing hemp extracts, and it is within the knowledge of one skilled in the art to apply such other known techniques in accordance with various embodiments of the present disclosure.

In at least some embodiments of the present disclosure, the hemp extract may be a resin comprising one or more cannabinoids, one or more flavonoids, and/or one or more terpenes. A bio extract of the present disclosure may be considered full spectrum in that it may contain all (or nearly all) of the non-intoxicating constituents of the cannabis (or hemp) dried inflorescence or plant material from which it was extracted.

### Whey Protein

Whey protein is a mixture of proteins (or group of globular proteins) present in whey (e.g., cheese whey), such as α-lactalbumin, β-lactoglobulin, serum albumin, lactoferrin, and immunoglobulins. Unless specifically indicated otherwise, as used herein “whey protein” may refer to whey protein concentrate (WPC) and/or whey protein isolate (WPI).

As used herein, “whey protein concentrate” and “WPC” refer to mixtures comprising at least 30 wt % whey protein. WPC may comprise at least about 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, 51 wt %, 52 wt %, 53 wt %, 54 wt %, 55 wt %, 56 wt %, 57 wt %, 58 wt %, 59 wt %, 60 wt %, 61 wt %, 62 wt %, 63 wt %, 64 wt %, 65 wt %, 66 wt %, 67 wt %, 68 wt %, 69 wt %, 70 wt %, 71 wt %, 72 wt %, 73 wt %, 74 wt %, 75 wt %, 76 wt %, 77 wt %, 78 wt %, 79 wt %, 80 wt %, 81 wt %, 82 wt %, 83 wt %, 84 wt %, 85 wt %, 86 wt %, 87 wt %, 88 wt %, or 89 wt % whey protein. In some embodiment, WPC may have at least 80 wt % whey protein. In some embodiments, WPC may have about 80 wt % to about 85 wt % whey protein.

As used herein, “whey protein isolate” and “WPI” refer to mixtures including at least about 90 wt % whey protein. WPI may comprise at least about 90 wt %, 91 wt %, 92 wt %, 93 wt %, 94 wt %, 95 wt %, 96 wt %, 97 wt %, 98 wt %, or 99 wt % whey protein. In some embodiments, WPI may include about 90 wt % to about 92 wt % whey protein.

### Polymerized Whey Protein

Polymerized whey protein is whey protein after its globular structures have been modified into relatively linear structures, and polymers have been formed (e.g., through thiol-disulphide interchange). Heat induced polymerized whey protein may have a hydrodynamic diameter up to 300 nm and similar properties to common food hydrocolloids and synthetic polymers. Various techniques for producing polymerized whey protein are known to those skilled in the art. Unless specifically indicated otherwise, polymerized whey protein of the present disclosure may be produced using any known or not yet discovered polymerization technique.

Polymerized whey protein of the present disclosure may be product from WPC, WPI, or a mixture of WPC and WPI.

### Compositions

A composition of the present disclosure may comprise hemp extract and a hemp extract encapsulate. The hemp extract may comprise at least CBD and terpenes. The hemp extract encapsulate may comprise β-cyclodextrin and at least one of whey protein and polymerized whey protein. As such, the hemp extract encapsulate may comprise β-cyclodextrin and one or more of WPC, WPI, a WPC/WPI mixture, polymerized WPC, polymerized WPI, and/or a mixture of polymerized WPC/WPI. In some embodiments, the hemp extract encapsulate may comprise one or more of treated WPC, treated WPI, and/or a mixture of treated WPC/WPI, where such treatment is other than polymerization.

Generally, the high lipophilicity and poor water solubility of CBD limit the absorption and bioavailability of CBD after consumption. β-cyclodextrin is a cone-shaped molecule having a hydrophobic cavity, and hydrophilic outer surface. β-cyclodextrin and CBD are sized such that CBD may be encapsulated within the hydrophobic cavity of β-cyclodextrin. By using β-cyclodextrin and at least one of whey protein and polymerized whey protein in the hemp extract encapsulate, both CBD and terpenes in the hemp extract may be encapsulated, thereby increasing the bioavailability of the CBD, and decreasing the “hempy” flavor provided by the terpenes.

In some embodiments, the hemp extract may include up to about 60 wt % CBD. In some embodiments, the hemp extract may include about 30 wt % to about 60 wt % CBD.

The hemp extract encapsulate may comprise the β-cyclodextrin, and the at least one of whey protein and polymerized whey protein, in different w/w ratios depending on intended use of the composition. For example, the β-cyclodextrin, and the at least one of whey protein and polymerized whey protein, may be present in a ratio of about 1:1 w/w, about 2:1 w/w, about 3:1 w/w, about 4:1 w/w, about 5:1 w/w, about 6:1 w/w, about 7:1 w/w, about 8:1 w/w, about 9:1 w/w, or about 10:1 w/w. In some embodiments, the β-cyclodextrin, and the at least one of whey protein and polymerized whey protein, may be present in a ratio of about 1:1 w/w. the β-cyclodextrin, and the at least one of whey protein and polymerized whey protein, may be present in a ratio of about 3:1 w/w. In some embodiments, the β-cyclodextrin, and the at least one of whey protein and polymerized whey protein, may be present in a range of about 3:1 w/w to about 5:1 w/w.

The composition may comprise hemp extract and hemp extract encapsulate in different w/w ratios depending on intended use of the composition. For example, hemp extract and hemp extract encapsulate may be present in a ratio of about 1:1 w/w, about 1:2 w/w, about 1:3 w/w, about 1:4 w/w, about 1:5 w/w, about 1:6 w/w, about 1:7 w/w, about 1:8 w/w, about 1:9 w/w, or about 1:10 w/w. In some embodiments, the hemp extract and hemp extract encapsulate may be present in a ratio of about 1:5 w/w.

### Consumable Compositions

A composition of the present disclosure may be formulated as, or included within, a consumable composition.

In some embodiments, the consumable composition may comprise a dairy component, meaning the consumable composition may comprise a component containing or made from milk. The present disclosure envisions use of milk secreted from various types of female mammals including, but not limited to, cows and goats,

In some embodiments, the consumable composition may be ice cream.

In some embodiments, the consumable composition may be yogurt.

In some embodiments, the consumable composition may be frozen yogurt.

In some embodiments, the consumable composition may be whipped cream.

In some embodiments, the consumable composition may be cheese.

In some embodiments, the consumable composition may be a powdered.

In some embodiments, the consumable composition may be a shake, in other words a blended drink containing high levels of protein and which may be consumed to, among other things, help the consumer gain muscle or weight or increase the consumer's level of energy.

In some embodiments, the consumable composition may be a tablet. In some embodiments, the consumable composition may be a capsule. As will be appreciated by one skilled in the art, a tablet may be formed at least in part from a composition of the present disclosure, whereas a capsule may include a shell within which a composition of the present disclosure is located. Various capsule shells are known and envisioned for use in accordance with the present disclosure.

In some embodiments, the consumable composition may be a food for a non-human animal. For example, the consumable composition may be a dog food, cat food, horse food, etc.

In some embodiments, encapsulation of hemp extract by a hemp extract encapsulate may occur as part of the process for preparing the consumable composition. In some embodiments, hemp extract may be encapsulated using a hemp extract encapsulate, and the encapsulated hemp extract may thereafter be used in the preparation of the consumable composition. In still other embodiments, hemp extract may be encapsulated using a hemp extract encapsulate, and the encapsulated hemp extract may thereafter be incorporated with an already prepared consumable composition.

It will be appreciated that the amounts of β-cyclodextrin, and whey protein and/or polymerized whey protein, may vary depending on the consumable composition and/or the desired characteristics thereof. For example, the more hemp flavor the consumable composition is to have, the less β-cyclodextrin may be used, and vice versa.

### Example Method of Making Ice Cream Comprising Hemp Extract

Teachings of the present disclosure may be used to make ice cream comprising hemp extract. The below is an example method for making such ice cream, although other methods of preparation are envisioned.

In an example method, milk may be prepared or obtained. The milk may be non-fat (i.e., skim) milk, reduced fat milk, or whole fat milk, as known in the industry. Preparation of the milk may include obtaining milk powder, and dissolving the milk powder in water.

The example method further comprises preparing a sugar syrup comprising stabilizer, emulsifier, and sugar in water.

A stabilizer is an ice cream ingredient that adds viscosity. The stabilizer may be one or more commercially available ice cream stabilizers. Generally, gums are considered to be powerful, flexible, and useful for use in ice cream. Example gums include, but are not limited to, guar gum, carob bean gum, and cellulose gum. In some embodiments, the stabilizer may comprise guar gum, carob bean gum, cellulose gum, and combinations thereof.

An emulsifier is an ice cream ingredient that “keeps everything together” over time, ensuring the texture of the ice cream remains constant. The emulsifier may be one or more commercially available ice cream emulsifiers. Example emulsifiers include, but are not limited to, egg yolk, soy lecithin, monogliceride, and digliceride. In some embodiments, the emulsifer may comprise egg yolk, soy lecithin, monogliceride, digliceride, and combinations thereof.

Generally, sugar is a sweet-tasting, soluble carbohydrate. The sugar, in the sugar syrup, may be any commercially available sugar. In some embodiments, the sugar may be a monosaccharide (sometimes referred to as a simple sugar). Example monosaccharides include, but are not limited to, glucose, fructose, and galactose.

The example method further comprises heating the sugar syrup to produce a heated sugar syrup. In some embodiments, producing the heated sugar syrup may comprise heating the sugar syrup to about 85° C.

The example method further comprises preparing a first mixture by mixing the milk with the heated sugar syrup.

The example method further comprises preparing a second mixture by mixing the first mixture with β-cyclodextrin.

The example method further comprises preparing or obtaining a third mixture comprising hemp extract and heavy cream. Heavy cream, sometimes referred to as heavy whipping cream, is the thick part of milk that rises to the top due to its high fat content. Heavy cream contains about 36% to about 40% fat.

The example method further comprises heating the third mixture to produce a heated third mixture. In some embodiments, producing the heated third mixture may comprise heating the third mixture to about 75° C.

The example method further comprises preparing a fourth mixture by mixing the heated third mixture with at least one of why protein or polymerized whey protein.

The example method further comprises producing the ice cream by mixing the second second mixture with the fourth mixture. In some embodiments, the whey protein/polymerized whey protein and β-cyclodextrin may be present in the ice cream in a ratio of about 1:3 w/w. In some embodiments, the whey protein/polymerized whey protein and β-cyclodextrin are present in the ice cream in a ratio of about 1:1 w/w. In some embodiments, the whey protein/polymerized whey protein and β-cyclodextrin may be present in the ice cream in a ratio of about 3:1 w/w to about 5:1 w/w. In some embodiments, the hemp extract may be present in the ice cream in a ratio of about 1:5 w/w with respect to the whey protein/polymerized whey protein and β-cyclodextrin.

### EXAMPLES

### Example 1. Preparation of Hemp Extract-Infused Ice Cream

### Section One: Ingredients and Equipment

Hemp extract was provided by Cattis Scientific.

Heavy cream (Hood®) and cane sugar (Domino®) were purchased from local grocery store.

Grade A low heat non-fat milk powder was purchased from Michigan milk.

Integrated mixture of stabilizer and emulsifier (MouldICE 160) was provided by Palsgaard.

Countertop ice cream machine (Model DF-200) was purchased from SaniServ.

Hand hold mixer was obtained from Cuisinart.

### Section Two: Making Hemp Extract-Infused Ice Cream

Decarboxylation of hemp extract: Hemp extract was heated at 155° C. for 2 h. One ml aliquot of sample was analyzed by HPLC to confirm that the cannabinoid-acid (CBD-A) was totally transferred to cannabinoid (CBD).

Preparation of polymerized whey protein (PWP): Whey protein solution (10%, w/v) was prepared by dissolving whey protein isolate (WPI) into water with constantly stirring for 1 h. Whey protein solution was stored at 4° C. overnight to totally hydrated. The next day, the pH was adjusted to 7.5 using 2 M sodium hydroxide at room temperature before heating at 85° C. for 30 min.

Preparation of hemp extract infused ice cream: Ice cream mix was formulated with 4.26% protein, 22.87% carbohydrates, 12.39% fat, and 0.55% mix of stabilizer and emulsifier (SE). To compare the flavor density with different amounts of hemp extract in ice cream, hemp extract was fortified in ice cream mix (3L) at a level of 0 g, 2 g, 4 g, 6 g, 8 g, and 10 g, separately. The amounts of ingredients are listed in Table 1 below.

TABLE 1
Ice cream ingredients and amounts.

| Ingredients | Amount | |
|---|---|---|
| Heavy cream | 1090 | mL |
| Cane sugar | 450 | g |
| Non-fat milk powder | 360 | g |
| Water | 1910 | mL |
| Stabilizer and emulsifier | 16.5 | g |

| | |
|---|---|
| Hemp extract | 0, 2 g, 4 g, 6 g, 8 g, 10 g |

Optimization of PWP and/or β-cyclodextrin level in hemp infused ice cream: Ice cream mix (3L) was prepared based on the above formulation with 8 g hemp extract. Rehydrated skim milk [360 g non-fat milk powder (NFMP)] was dissolved with 1000 mL water. 16.5 g mixture of stabilizer and emulsifier (SE) was pre-mixed with 360 g sugar before dissolving in 910 mL water. Then the sugar syrup was heated at 85° C. for 5 min. The heated sugar syrup was added into the rehydrated skim milk and mixed well. The hemp extract and heavy cream were warmed up in a 75° C. water bath. 8 g hemp extract was weighted and dissolved in 1090 mL heavy cream. As represented in Table 2 below, the amount of PWPI and/or β-cyclodextrin was separately added in ice cream mix with hemp extract.

TABLE 2
Experimental Design

| | Hemp extract encapsulate (g) | | | Hemp |
|---|---|---|---|---|
| Sample # | WPI | PWPI | β-cyclodextrin | extract (g) |
| 1 (Plain) | 0 | 0 | 0 | 0 |
| 2 (Control) | 0 | 0 | 0 | 8 |
| 3 | 40 | 0 | 0 | 8 |
| 4 | 0 | 40 | 0 | 8 |
| 5 | 0 | 0 | 29.5 | 8 |
| 6 | 0 | 10 | 30 | 8 |
| 7 | 0 | 20 | 20 | 8 |
| 8 | 0 | 30 | 10 | 8 |

β-cyclodextrin was dispersed into the rehydrated skim milk with the sugar syrup.

PWPI and WPI were added into the heavy cream with hemp extract.

The rehydrated skim milk mixture was mixed with the heavy cream by a hand hold mixer at high speed (30 s, 3×).

The ice cream mix was pasteurized at 75° C. for 10 min and rapidly cooled down to room temperature.

The final ice cream mix was stored at 4° C. overnight for aging purposes.

The next day, the ice cream mix was removed from the refrigerator and mixed well before making ice cream by an ice cream machine.

All the samples were labeled and stored in a freezer for later analyses.

### Example 2. Sensor Evaluation Analysis of Hemp-Infused Ice Cream of Example 1

Panelists within a wide range of age and from both sexes participated in this study. The panelists were asked to score eight attributes of the ice cream: hemp flavor (selective odor representatives of spice-like, pine-like, and bitterness), milky flavor (perception of the flavor of rehydrated milk), buttery flavor (perception of the flavor of butter), sweetness (the strength of the sweetness from mild to strong), firmness (the force necessary to compress the sample by the tongue against the roof of the mouth), slipperiness (the amount in which the sample slides across the tongue), creaminess (a combination of thickness and lubricative feeling as the ice cream melst), and mouth coating (residual perceived on oral surfaces after sample is swallowed). A 10 point hedonic scale was used, ranging varying from 0 “not significant” to 9 “extremely highest”, to score each flavor attribute. All samples were served in 8-oz containers with random labels. The samples were taken from the freezer 10 min before tasting. The panelists were given 4-5 samples including a plain flavor sample as a reference. Before tasting each sample, the panelists were asked to wash out their mouth using warm water.

Results:

The polymerized whey protein (PWP) had better flavor masking effect than native whey protein isolate (WP).

Both PWP and β-cyclodextrin reduced hempy flavor. Samples tasted less bitter, spicy, and piney than the sample only with hemp extract, but samples with PWP or β-cyclodextrin still had odors of hemp extract.

Samples containing both PWPI and β-cyclodextrin had better flavor masking effects than the individuals. Compared with the samples with either PWP or β-cyclodextrin, the samples with both did not have hempy odors detected by the panelists.

The ratio of flavor masking agents (i.e., β-cyclodextrin and PWP or WP) to hemp extract was 5:1. In addition, a 1:3 ratio of PWP to β-cyclodextrin had better flavor masking effects than other combinations.

### Example 3. Impact of Polymerized Whey Protein on the Microstructure, Probiotic Survivability, and Sensory Properties of Goat Milk Yogurt Infused With Hemp Extract

### Section 1: Materials and Methods

Goat milk was purchased from Oak Knoll, Vermont. Whole cow milk was purchased from monument farmer, Vermont. Sugar was purchased from a local grocery store. Whey protein isolate was bought from Fonterra, New Zealand. Full spectrum hemp extract was provided by Cattis Scientific, Vermont. Starter cultures (ABY-3) were provided by Chr. Hansen, Maryland, U.S., consisting of Streptococcus thermophilus, Lactobacillus delbrueckii subsp. bulgaricus, Bifidobacterium BB-12, and Lactobacillus acidophilus LA-5.

### Section 2: Preparation of Whey Protein Polymerization

According to Wang, W., Bao, Y., Hendricks, G. M., & Guo, M. (2012). Consistency, microstructure and probiotic survivability of goats' milk yogurt using polymerized whey protein as a co-thickening agent. International Dairy Journal, 24(2), 113-119. https://doi.org/10.1016/j.idairyj.2011.09.007, whey protein isolate (WPI, 10% w/v) solution was prepared at room temperature and fully hydrated at 4° C. overnight. Next day, WPI was warmed up to room temperature and the pH was adjusted to 7.5 using 2 M sodium hydroxide. Then the WPI solution was heated at 85° C. for 30 min and rapidly cool down using cold water.

### Section 3: Yogurt Manufacturing

Raw hemp extract was decarboxylated by heating at 155° C. for 2 hours to transfer the acid form of cannabinoids to its neutral form. Four groups with different treatments for goat yogurts were made as follows: plain, hemp extract (HE) infused, HE infused with WPI (0.5%, v/v), and HE infused with PWP (0.5%, v/v). The yogurts fortified with HE were designed to contain 25 mg CBD per container (160 mL). Hemp extract oil was melted in boiling water and then emulsified with WPI or PWP by high-speed blending. Goat milk was mixed with sugar (6%), with or without HE, WPI, and PWP based on the above formulation. A hand hold blender was used to mix all the ingredients together at high speed. Then the goat milks were pasteurized at 80° C. water bath for 20 min and cooled down to 45° C. before inoculating with 0.1% w/v ABY-3 starter cultures. Next, the samples were poured into cups (160 mL) and incubated at 43° C. until the pH dropped to 4.5. Cow yogurts were made with the same formulation as the reference. All the yogurt samples were stored at 4° C. for further analysis. Three trials of yogurts were produced on three different days.

### Section 4: Physio-Chemical Analysis

pH was tested by a pH meter (Apera Instruments, LLC., Columbus, Ohio, USA) once a week at room temperature. The viscosity was analyzed weekly by a Brookfield DV-I prime viscometer (Brookfield Engineering Laboratories, Inc., Middleboro, MA, USA) and expressed in mPa.s. The viscosity was measured with no.3 spindle at 50 rpm for 30 s. Titratable acid was analyzed by 0.1 N standard sodium hydroxide. The yogurt samples were analyzed for total solids, protein, fat, and ash contents using standard Association of Official Analytical Chemists procedures (AOAC, 2002). The content of carbohydrates was calculated by the difference of total solids minus other solid components. All the tests were triplicated, and the results were expressed as mean with standard derivation.

### Section 5: Probiotic Survivability Test

The measurement of probiotic survivability was conducted weekly according to the procedures of Chr. Hansen (2005). Lactobacillus acidophilus and Bifidobacterium in fermented milk products-Guildelines and method for counting probiotic bacteria. Ten grams of samples were taken from yogurts and put in 99 mL peptone dilution water. Having been diluted in a series of peptone water, one mL (from 10⁻⁷, 10⁻⁸, 10⁻⁹) was transferred to the sterile petri dishes. MRS (De Man, Rogosa and Sharpe) agar with L-cysteine, lithium chloride, and dicloxacillin was used to anaerobically enumerate BB-12 at 37° C. for 72 h. Lactobacillus acidophilus LA-5 was selectively grown on MRS with clindamycin at 37° C. in an anaerobic environment for 72 h.

### Section 6: Scanning Electron Microscopy

The microstructure of the yogurt sample was analyzed by JSM 6060 scanning electron microscope (JEOL USA, Inc, Peabody, MA). The method was followed by the study of Walsh, H., Ross, J., Hendricks, G., & Guo, M. (2010). Physico-Chemical Properties, Probiotic Survivability, Microstructure, and Acceptability of a Yogurt-Like Symbiotic Oats-Based Product Using Pre-Polymerized Whey Protein as a Gelation Agent. Journal of Food Science, 75(5), M327-M337. https://doi.org/10.1111/j.1750-3841.2010.01637.x, with slight modification. Yogurt samples were prepared in the agarose disk. Sections of yogurt samples were fractured with a blade and fixed in Karnovsky's solution overnight. The next day, the samples were rinsed by cacodylate Buffer (pH 7.2) and stored in cacodylate Buffer overnight. Next, after the samples were dehydrated in a series of ethanol to remove extra water, they were snap-frozen in liquid nitrogen and followed by critical point drying with liquid carbon dioxide. The fragments were mounted on aluminum SEM stubs and coated with gold and palladium by vacuum evaporation. The SEM was operated at 20 kV (×5500 resolution).

### Section 7: Sensor Evaluation

The University of Vermont Institutional Review Board approved the study (STUDY00002058). Participants were recruited from the University of Vermont using posted research study flyers. Participants were screened for age, allergies, and willingness to participate. Twenty qualified participants were selected if they were 21 years old, did not have any known food allergies, and were able to consume and taste dairy products and hemp extract containing cannabinoids. A consent form containing information about the study and its purpose was signed before the start of the study. Then, participants were trained on how to taste the samples and score the flavor attributes on the sensory evaluation form. Each participant received 8 yogurt samples and one sensory evaluation form. Water and plain crackers were provided to clean out the palate between each sample. The study was conducted on the campus of the University of Vermont. Descriptive analysis and check-all-that-apply (CATA) were used in the study with some modifications (Alqahtani, N. K., Darwish, A. A., El-Menawy, R. K., Alnemr, T. M., & Aly, E. (2021). Textural and organoleptic attributes and antioxidant activity of goat milk yogurt with added oat flour. International Journal of Food Properties, 24(1), 433-445. https://doi.org/10.1080/10942912.2021.1900237). Participants were asked to score their liking of the hemp extract flavor, goaty flavor, consistency, aftertaste, and overall liking on 5-point hedonic scales ranging from 1=Dislike extremely to 5=Like extremely. After scoring the samples on the 5-point hedonic scales, the participants were asked to answer a CATA questionnaire including 5 attributes such as creamy, sweet, acid, milk/dairy flavor, and gummy.

### Section 8: Statistical Analysis

All experiments were carried out in three replications. Q-Q plot and Shapiro test were used to evaluate the normal distribution of data. Results were analyzed using a mixed-effect model. The significance of differences between mean values was estimated by the Tukey post-hoc test, at a p<0.05. The standard deviation (±SD) was determined for all reported mean values. The statistical analysis was performed using R version 4.2.2.

### Section 9: Results and Discussion

### Section 9a: The Physio-Chemical Analyses

TABLE 3
Chemical composition of goat milk yogurt and cow milk yogurt.

| Composition | Control | | HE | | HE + WPI | | HE + PWP | |
|---|---|---|---|---|---|---|---|---|
| (%) | GY | CY | GY | CY | GY | CY | GY | CY |
| Total solid | 18.23 ± 1.11 | 18.04 ± 1.94 | 17.78 ± 1.92 | 19.57 ± 0.37 | 17.60 ± 2.10 | 17.06 ± 2.13 | 19.22 ± 1.36 | 19.56 ± 0.37 |
| Fat | 2.90 ± 0.18 | 3.40 ± 0.25 | 2.92 ± 0.40 | 3.39 ± 0.15 | 2.68 ± 0.25 | 3.21 ± 0.19 | 2.84 ± 0.16 | 3.49 ± 0.14 |
| Protein | 3.64 ± 0.06 | 3.23 ± 0.42 | 3.44 ± 0.43 | 3.45 ± 0.21 | 3.53 ± 0.45 | 3.79 ± 0.11 | 3.05 ± 0.11 | 4.10 ± 0.51 |
| Carbohydrates | 10.81 ± 0.97 | 10.71 ± 1.83 | 10.68 ± 2.09 | 12.05 ± 0.16 | 10.52 ± 2.85 | 10.32 ± 2.32 | 12.55 ± 1.12 | 11.30 ± 0.31 |
| Ash | 0.87 ± 0.07 | 0.70 ± 0.03 | 0.72 ± 0.09 | 0.67 ± 0.02 | 0.87 ± 0.16 | 0.65 ± 0.001 | 0.77 ± 0.12 | 0.66 ± 0.02 |

GY: goat milk yogurt; CY: cow milk yogurt; HE: hemp extract; WPI: whey protein isolate; PWP: polymerized whey protein.

The chemical compositions of goat milk yogurts and cow milk yogurts of this Example 3 are shown in Table 3. The chemical composition of yogurts varied depending on the milk type, manufacturing, and fortification process. The addition of PWP and hemp extract did not significantly change the chemical composition of both goat milk yogurt and cow milk yogurt; however, the fat content in goat milk yogurt is lower than cow milk yogurt and the amount of ash in goat yogurt is higher than cow yogurt (p<0.05).

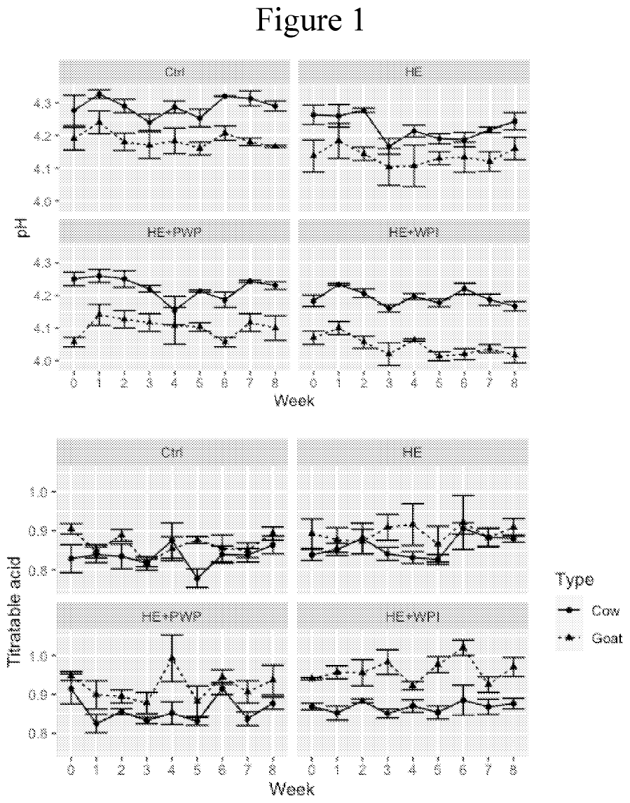

As illustrated in FIG. 1, the changes in pH in goat milk yogurt were relatively stable during the shelf-life test in each group. The pH of goat milk yogurt was 0.1-0.2 units lower than cow milk yogurts, which echoed the results in the titratable acid of goat milk, which was higher than that of cow milk. Compared with the control groups, adding WPI, PWP, and HE significantly reduced the pH and increased the titratable acid of both goat and cow milk yogurt (p<0.001). The viscosity of goat milk was lower than cow milk in each group (p<0.05). Compared with the control, the addition of PWP increased around two times of viscosity in goat yogurt, which the viscosity of goat milk yogurt with PWP and HE increased to 88.11±29.57 mPa·S from 25.76±14.07 mPa·S in control. The cow yogurt showed a similar trend. The lower content and different composition of caseins in goat milk cause a more fragile clot and lower apparent viscosity in goat milk yogurts than cow milk yogurt (Costa, M. P., Monteiro, M. L. G., Frasao, B. S., Silva, V. L. M., Rodrigues, B. L., Chiappini, C. C. J., & Conte-Junior, C. A. (2017). Consumer perception, health information, and instrumental parameters of cupuassu (Theobroma grandiflorum) goat milk yogurts. Journal of Dairy Science, 100(1), 157-168. https://doi.org/10.3168/jds.2016-11315). For example, in this study, the viscosity of goat and cow milk yogurt control group was 25.76±14.07 mPa·S and 57.58±24.88 mPa·S, respectively. The addition of PWP resulted in a more rigid gel structure in yogurt due to the formation of aggregates via interactions between PWP and casein micelles (Andoyo, R., Guyomarc'h, F., Cauty, C., & Famelart, M.-H. (2014). Model mixtures evidence the respective roles of whey protein particles and casein micelles during acid gelation. Food Hydrocolloids, 37, 203-212. https://doi.org/10.1016/j.foodhyd.2013.10.019). The titratable acid of goat milk yogurt was higher than cow milk yogurt, which may be due to the fatty acid composition of goat milk that is different from cow milk. Goat milk was characterized by the high content of medium-and short-chain fatty acids, like caproic, caprylic, and capric acid, which could increase the titratable acidity (Azizkhani, M., Saris, P. E. J., & Baniasadi, M. (2021). An in-vitro assessment of antifungal and antibacterial activity of cow, camel, ewe, and goat milk kefir and probiotic yogurt. Journal of Food Measurement and Characterization, 15(1), 406-415. https://doi.org/10.1007/s11694-020-00645-4).

### Section 9b: Probiotic Survivability

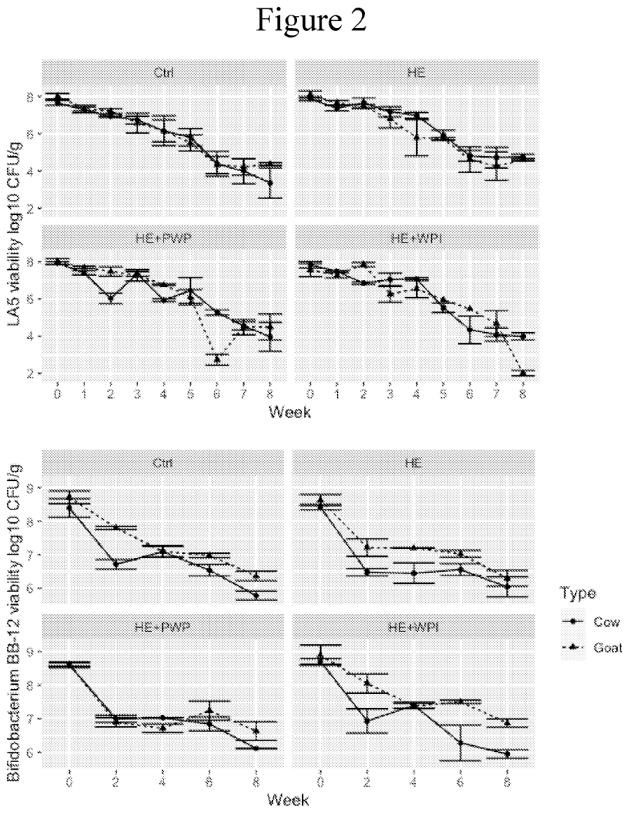

FIG. 2 includes plots showing changes of Lactobacillus acidophilus LA-5 and Bifidobacterium BB-12 in both goat and cow milk yogurt during storage.

The initial amount of Lactobacillus acidophilus LA-5 in fresh cow and goat yogurt was about 10⁸ cfu/g and was relatively stable at the first 5 weeks; then, it quickly dropped below 10⁴ cfu/g. The amount of BB-12 was about 10⁸ cfu/g in fresh goat and cow milk yogurt. With a sharp drop in the first two weeks, the viability of BB-12 was maintained at around 10⁶ cfu/g in the remaining weeks. After 8 weeks of storage, the BB-12 in goat yogurt maintained higher viability than in cow yogurt. The existing Lactobacillus bulgaricus could produce lactic acid after fermentation, known as post-acidification, which decreased in pH and also produced hydrogen peroxide during refrigerated storage. These byproducts can affect the viability of Lactobacillus acidophilus LA-5 in goat milk yogurt (Menezes, M. U. F. O., Bevilaqua, G. C., Ximenes, G. N. da C., Andrade, S. A. C., Kasnowski, M. C., & Barbosa, N. M. dos S. C. (2022). Viability of Lactobacillus acidophilus in whole goat milk yogurt during fermentation and storage stages: A predictive modeling study. Food Science and Technology, 42, e50922. https://doi.org/10.1590/fst.50922). BB-12 showed better survivability than L. acidophilus LA-5 during the storage of goat milk yogurt, similar results were also reported by Wang, W., Bao, Y., Hendricks, G. M., & Guo, M. (2012). Consistency, microstructure and probiotic survivability of goats milk yogurt using polymerized whey protein as a co-thickening agent. International Dairy Journal, 24(2), 113-119. https://doi.org/10.1016/j.idairyj.2011.09.007. They indicated that S. thermophilus could be beneficial for the growth and survival of Bifidobacterium spp. as an oxygen scavenger creating an anaerobic environment.

### Section 9c: Microstructure

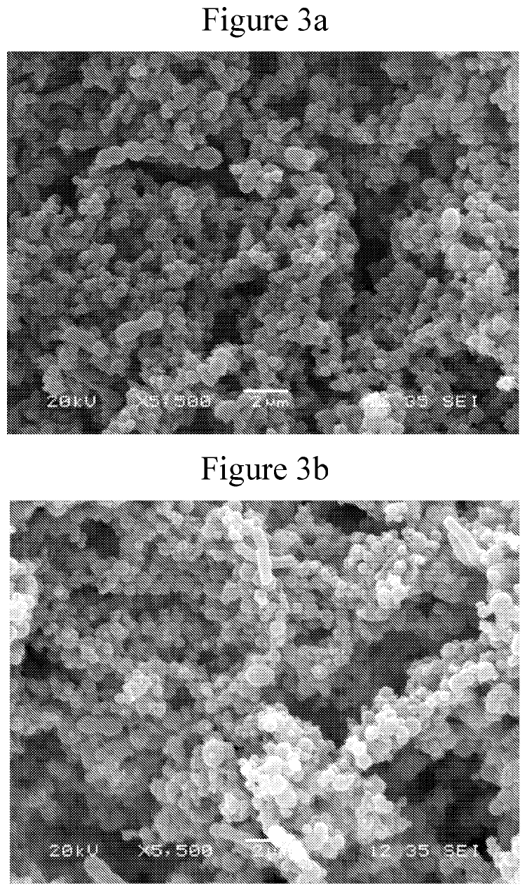

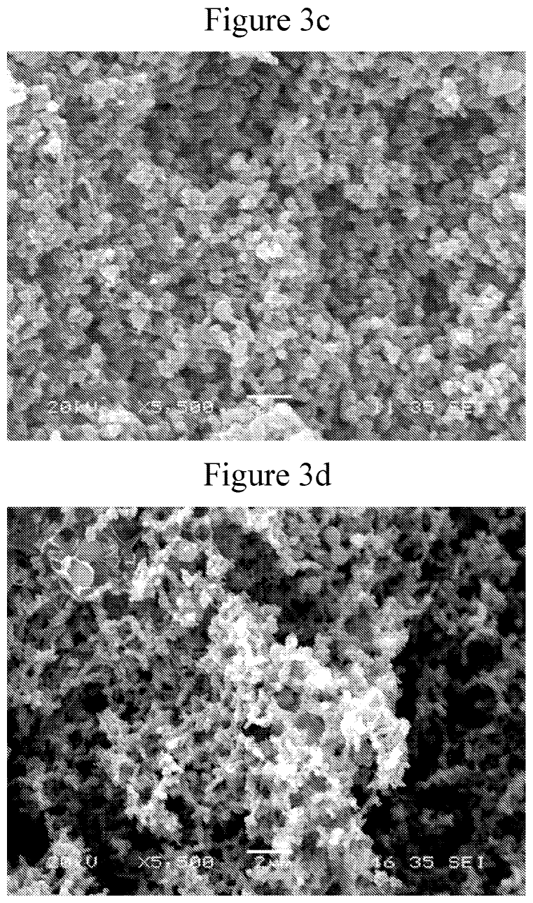

The microstructure of yogurts was observed using a scanning electron microscope (SEM) at a magnification of 5500. As shown in FIGS. 3a through 3d, the microstructure of all the yogurt samples were well-defined three-dimensional networks filled with combined probiotics and yogurt starter cultures. The presence of lactic bacteria, Streptococcus thermophiles and Lactobacillus burglaries, and Bifidobacterium BB-12, Lactobacillus acidophilus LA-5 were easily observed in all the micrographs, which indicated that the probiotics strains grew well in yogurts and could maintain in a good number before exerting their health benefits to the host. In addition, the SEM micrographs of FIGS. 3a through 3d illustrate the differences in the microstructure of the yogurts such as particles and pore sizes, which provides a deeper insight into flavors and textural properties. The micrographs of FIGS. 3a and 3b consisted of relatively loose large particles interspaced by voids that were originally entrapped by whey. The void spaces were larger and unevenly distributed in the protein matrix. Milk-fat globules were integrated into the protein matrix of the yogurts. The addition of WPI into HE-infused goat yogurts (see FIG. 3c) did not form a gel networking compared with the effect of PWP (see FIG. 3d), but the structure became more condensed and more compact compared with plain flavor and HE-infused yogurts (see FIGS. 3a and 3b). With the addition of PWP, the goat milk yogurt (see FIG. 3d) was characterized by the honeycomb-like and most interconnected fine-pored gel structure with the smallest particle and pore size, which indicate a better immobilization of water. The improved gel structure of goat milk yogurt could be contributed to the gelation property of the PWP (L. i, Jiancai & Guo, M. (2006). Effects of Polymerized Whey Proteins on Consistency and Water-holding Properties of Goat's Milk Yogurt. Journal of Food Science, 71(1), C34-C38. https://doi.org/10.1111/j.1365-2621.2006.tb12385.x; Wang, W., Bao, Y., Hendricks, G. M., & Guo, M. (2012). Consistency, microstructure and probiotic survivability of goats' milk yoghurt using polymerized whey protein as a co-thickening agent. International Dairy Journal, 24(2), 113-119. https://doi.org/10.1016/j.idairyj.2011.09.007). In the heat treatment (above 70° C.), whey protein partially unfolded and exploded the hydrophobic residues and free sulfhydryl groups, which increased the possibility of hydrophobic attractions and the formation of disulfide bonds between whey proteins. It may also result in increased aggregation of whey protein molecules. When pH is lowered to the isoelectric point of β-lactoglobulin, a major protein in whey protein, during goat milk fermentation, the electrostatic repulsion tends to decrease and consequently traps more water and other components in the network. This process is also known as acid-induced cold-set gelation (Alting, A. C., van der Meulena, E. T., Hugenholtz, J., & Visschers, R. W. (2004). Control of texture of cold-set gels through programmed bacterial acidification. International Dairy Journal, 14(4), 323-329. https://doi.org/10.1016/j.idairyj.2003.09.008).

### Section 9d: Sensory Evaluation (In Progress)

Preliminary results from five participants showed that the addition of PWP could reduce the hempy and goaty flavor and increased the overall likeness of hemp extract-infused goat yogurt.

### Section 10: Conclusions

Goat milk yogurt infused with hemp extract is a good carrier to deliver probiotics. The survival rates of Bifidobacterium BB-12 remained at above 10⁶ cfu/g over the 8 weeks of storage. Lactobacillus acidophilus LA-5 maintained viable counts at around 10⁶ cfu/g in the first 5 weeks and then decreased sharply. From the data of microstructure and viscosity analyses, the addition of PWP improved the gel structure of goat milk yogurt. Fortification of PWP also improved the sensory properties of hemp extract-infused goat milk yogurt.

### Further Definitions and Construction of Terms

The titles, headings, and subheadings provided herein should not be interpreted as limiting the various aspects of the disclosure. Accordingly, the terms defined herein are more fully defined by reference to the specification in its entirety. All references cited herein are incorporated by reference in their entirety.

Unless otherwise defined, scientific and technical terms used herein shall have the meanings that are commonly understood by those of ordinary skill in the art. Further, unless otherwise required by context, singular terms shall include pluralities, and plural terms shall include the singular.

In this application, the use of “or” means “and/or” unless stated otherwise. In the context of a multiple dependent claim, the use of “or” refers back to more than one preceding independent or dependent claim in the alternative only.

It is further noted that, as used in this specification and the appended claims, the singular forms “a,” “an,” and “the,” and any singular use of any word, include plural referents unless expressly and unequivocally limited to one referent.

As used herein, the term “about,” means approximately. When the term “about” is used in conjunction with a numerical range, it modifies that range by extending the boundaries above and below the numerical values set forth. Illustratively, the use of the term “about” indicates that values slightly outside the cited values (i.e., plus or minus 0.1% to 10%), which are also effective and safe are included in the value. Numerical ranges recited herein by endpoints include all numbers and fractions subsumed within that range (e.g. 1 to 5 includes 1, 1.5, 2, 2.75, 3, 3.90, 4, and 5).

As used herein, the terms “comprising” (and any form of comprising, such as “comprise,” “comprises,” and “comprised”), “having” (and any form of having, such as “have” and “has”), “including” (and any form of including, such as “includes” and “include”), and “containing” (and any form of containing, such as “contains” and “contain”) are inclusive or open-ended and do not exclude additional, un-recited elements or method steps. Additionally, a term that is used in conjunction with the term “comprising” is also understood to be able to be used in conjunction with the term “consisting of” or “consisting essentially of.”

Method steps described in this disclosure can be performed in any order unless otherwise indicated or otherwise clearly contradicted by context.

For the avoidance of doubt, insofar as is practicable any embodiment of a given aspect of the present disclosure may occur in combination with any other embodiment of the same aspect of the present disclosure. In addition, insofar as is practicable it is to be understood that any preferred or optional embodiment of any aspect of the present disclosure should also be considered as a preferred or optional embodiment of any other aspect of the present disclosure.

1. A composition comprising:
hemp extract comprising cannabidiol (CBD) and terpenes; and
hemp extract encapsulate comprising β-cyclodextrin and at least one of whey protein and polymerized whey protein.

2. The composition of claim 1, wherein the β-cyclodextrin and whey protein/polymerized whey protein are present in a ratio of about 3:1 w/w.

3. The composition of claim 1, wherein the β-cyclodextrin and whey protein/polymerized whey protein are present in a ratio of about 1:1 w/w.

4. The composition of claim 1, wherein the whey protein/polymerized whey protein and β-cyclodextrin are present in a ratio of about 3:1 w/w to about 5:1 w/w.

5. The composition of claim 1, wherein the hemp extract and hemp extract encapsulate are present in a ratio of about 1:5 w/w.

6. The composition of claim 1, wherein the β-cyclodextrin and whey protein/polymerized whey protein are configured to increase bioavailability of the CBD.

7. The composition of claim 1, wherein the β-cyclodextrin and whey protein/polymerized whey protein are configured to mask the flavor of the terpenes.

8. The composition of claim 1, wherein the hemp extract comprises about 30 wt % to about 60 wt % CBD.

9. The composition of claim 1, wherein the hemp extract encapsulate comprises at least one of whey protein isolate and polymerized whey protein isolate.

10. A consumable composition comprising the composition of claim 1.

11. The consumable composition of claim 10, further comprising a dairy component.

12. The consumable composition of claim 11, wherein the consumable composition is selected from the group consisting of ice cream, yogurt, frozen yogurt, whipped cream, and cheese.

13. The consumable composition of claim 10, wherein the consumable composition is selected from the group consisting of a powder, a shake, a tablet, and a capsule.

14. The consumable composition of claim 10, wherein the consumable composition is dog food.

15. A method of preparing ice cream comprising hemp extract, the method comprising:
preparing or obtaining milk;
preparing a sugar syrup comprising stabilizer, emulsifier, and sugar in water;
heating the sugar syrup to produce a heated sugar syrup;
preparing a first mixture by mixing the milk with the heated sugar syrup;
preparing a second mixture by mixing the first mixture with β-cyclodextrin;
preparing or obtaining a third mixture comprising hemp extract and heavy cream;
heating the third mixture to produce a heated third mixture;
preparing a fourth mixture by mixing the heated third mixture with at least one of whey protein and polymerized whey protein; and
producing the ice cream mixture by mixing the second mixture with the fourth mixture.

16. The method of claim 15, wherein preparing the milk comprises dissolving milk powder in water.

17. The method of claim 15, wherein the stabilizer comprises guar gum, carob bean gum, cellulose gum, and combinations thereof.

18. The method of claim 15, wherein the emulsifer comprises egg yolk, soy lecithin, monogliceride, digliceride, and combinations thereof.

19. The method of claim 15, wherein producing the heated sugar syrup comprises heating the sugar syrup to about 85° C.

20. The method of claim 15, wherein producing the heated third mixture comprises heating the third mixture to about 75° C.

21-24. (canceled)
